(19) United States
(12) Patent Application Publication
(10) Pub. No.: US 2021/0378722 A1
(43) Pub. Date: Dec. 9, 2021

(54) BOWED PLATE FOR FIXATING OSTEOTOMIES AND JOINT ARTHRODESES

(71) Applicant: In2Bones USA, LLC, Memphis, TN (US)

(72) Inventors: Alan G. Taylor, Memphis, TN (US); Rebecca Hawkins Wahl, Escondido, CA (US); Bradley Cowan, Memphis, TN (US)

(73) Assignee: In2Bones USA, LLC, Memphis, TN (US)

(21) Appl. No.: 17/341,811

(22) Filed: Jun. 8, 2021

Related U.S. Application Data
(60) Provisional application No. 63/036,328, filed on Jun. 8, 2020.

(51) Int. Cl. A61B 17/80; A61B 17/88

(52) U.S. Cl. CPC A61B 17/8014; A61B 17/8057; A61B 2017/564; A61B 17/808; A61B 17/8863

(57) ABSTRACT

An apparatus and methods are provided for a bowed plate for fixating osteotomies and joint arthrodeses. The bowed plate includes a backplate comprising a support portion and an aperture portion that share an intervening central portion. The central portion angles the support portion and the aperture portion toward a midline of the bowed plate. A leg extends from the support portion and includes fixation features that enable the leg to grip an inner wall of a hole drilled in bone. A slanted aperture in the aperture portion is configured to countersink a bone screw. The central portion is configured to be straightened by way of a surgical bending instrument and implanted at a bone fixation or fusion site of a patient. Upon relaxing the surgical bending instrument, the leg and the bone screw compress the fusion site.

### PRIORITY

This application claims the benefit of and priority to U.S. Provisional application, entitled “Bowed Plate For Fixating Osteotomies And Joint Arthrodeses,” filed on Jun. 8, 2020 and having application Ser. No. 63/036,328, the entirety of said application being incorporated herein by reference.

### FIELD

Embodiments of the present disclosure generally relate to securing bones together. More specifically, embodiments of the disclosure relate to an apparatus and methods for a bowed plate for fixating osteotomies and joint arthrodeses.

### BACKGROUND

Surgical staples or a fusion bone plate implant utilized in conjunction with one or more fasteners may be used to generate compression and stability at a bone interface. An implant generally serves to stabilize bones, or bone parts, relative to one another so as to promote bone fusion. In many applications, surgical staples, or bone plates and fasteners are used to fuse bones, or bone parts, of the human body, such as bones in the foot, the ankle, the hand, the wrist, as well as various other portions of the body. Surgical staples are particularly advantageous in the hands and feet due to a low dorsal profile of the staples once they are implanted. Furthermore, during the course of certain medical procedures, a surgeon may immobilize one or more bones or bone fragments by stabilizing the bones together in a configuration which approximates the natural anatomy. To this end, the surgeon may use fasteners to attach the bones to a bone plate implant so as to hold the bones in alignment with one another while they fuse together.

### SUMMARY

An apparatus and methods are provided for a bowed plate for fixating osteotomies and joint arthrodeses. The bowed plate includes a backplate comprising a support portion and an aperture portion that share an intervening central portion. The central portion angles the support portion and the aperture portion toward a midline of the bowed plate. A leg extends from the support portion and includes fixation features that enable the leg to grip an inner wall of a hole drilled in bone. A slanted aperture in the aperture portion is configured to countersink a bone screw. The central portion is configured to be straightened by way of a surgical bending instrument and implanted at a bone fixation or fusion site of a patient. Upon relaxing the surgical bending instrument, the leg and the bone screw compress the fusion site.

In an exemplary embodiment, a bowed plate for fixating osteotomies and joint arthrodeses comprises: a backplate comprising a support portion and an aperture portion that share an intervening central portion; a leg extending from the support portion; and one or more fixation features disposed on the leg.

In another exemplary embodiment, the one or more fixation features include one or more teeth, circumferential grooves, threads, or any combination thereof. In another exemplary embodiment, the bowed plate is comprised of a material that exhibits elastic properties, such as Nitinol or other metallic or polymer materials. In another exemplary embodiment, the leg is adapted to be implanted into a hole drilled in a bone. In another exemplary embodiment, the one or more fixation features are configured to enable the leg to grip an inner wall of a bone hole.

In another exemplary embodiment, the central portion includes a curvature that angles the leg and a centerline of the slanted aperture toward a midline of the bowed plate. In another exemplary embodiment, the central portion is configured to be straightened by way of a surgical bending instrument for placement at a bone fixation or fusion site of a patient. In another exemplary embodiment, the central portion includes a first notch and a second notch that are adapted to respectively accommodate a first grip and a second grip comprising a surgical bending instrument for straightening the bowed plate.

In another exemplary embodiment, the first notch and the second notch serve to center the bowed plate within the surgical bending instrument and enable the first grip and the second grip to extend across a majority of the width of the backplate. In another exemplary embodiment, the central portion is configured to facilitate removing the first grip and the second grip from between a bone surface and the backplate after implantation of the bowed plate into the bone. In another exemplary embodiment, the central portion is bound by a first guide edge and a second guide edge that cooperate to center the bowed plate between the first grip and the second grip of the surgical bending instrument. In another exemplary embodiment, the first notch and the second notch and the first guide edge and the second guide edge are configured to position a driver of the surgical bending instrument at a location of the central portion that is optimal for straightening the bowed plate.

In an exemplary embodiment, a bowed plate for fixating osteotomies and joint arthrodeses comprises: a backplate comprising a central portion having a first end and a second end; a support portion joined with the first end; a first aperture portion joined with the second end; and a second aperture portion joined with the second end.

In another exemplary embodiment, a leg extends from the support portion and includes one or more teeth, circumferential grooves, threads, or any combination thereof. In another exemplary embodiment, each of the first aperture portion and the second aperture portion includes a slanted aperture for receiving a bone screw. In another exemplary embodiment, the support portion comprises a third aperture portion that includes a slanted aperture for receiving a bone screw.

In an exemplary embodiment, a bowed plate assembly for fixating osteotomies and joint arthrodeses comprises: a central portion disposed between a first aperture portion and a second aperture portion; one or more legs coupled with the first aperture portion; and one or more legs coupled with the second aperture portion.

In another exemplary embodiment, any one of the one or more legs includes one or more circumferential teeth that are biased toward the central portion. In another exemplary embodiment, any one of the one or more legs comprises a support portion that is adapted to be threaded into a threaded aperture disposed within the first aperture portion or the second aperture portion. In another exemplary embodiment, the support portion includes a flat top surface surrounded by tapered peripheral threads that are configured to engage with threads comprising the threaded aperture. In another exemplary embodiment, the first aperture portion includes side-by-side aperture portions. In another exemplary embodiment, the second aperture portion includes one or more in-line aperture portions.

In an exemplary embodiment, a method for fixating a bone fusion site of a patient comprises: drilling a first bone hole at the bone fusion site; drilling a second bone hole across the bone fusion site; straightening a bowed plate by way of a surgical bending instrument; inserting a leg comprising the bowed plate into the first bone hole; extending a bone screw through an aperture portion of the bowed plate; tightening the bone screw into the second bone hole; relaxing the bowed plate such that the leg and the bone screw compress the bone fusion site; and removing the surgical bending instrument from between the bone fusion site and the bowed plate.

In another exemplary embodiment, inserting the leg includes aligning a slanted aperture with the second bone hole. In another exemplary embodiment, tightening includes causing the aperture portion and a support portion to contact the bone fusion site. In another exemplary embodiment, relaxing includes reducing a degree of force applied to the bowed plate by the surgical bending instrument. In another exemplary embodiment, straightening includes: loading the bowed plate into the surgical bending instrument; activating the surgical bending instrument to forcibly clasp the bowed plate; and operating the surgical bending instrument to manipulate the bowed plate from a bowed configuration to a straightened configuration.

These and other features of the concepts provided herein may be better understood with reference to the drawings, description, and appended claims.

### BRIEF DESCRIPTION OF THE DRAWINGS

The drawings refer to embodiments of the present disclosure in which:

FIG. 1 illustrates an isometric view of an exemplary embodiment of a bowed plate in accordance with the present disclosure;

FIG. 2 illustrates top plan view of the exemplary embodiment of the bowed plate of FIG. 1;

FIG. 3 illustrates side plan view of the exemplary embodiment of the bowed plate of FIG. 2;

FIG. 4 illustrates an exemplary embodiment of a bowed plate that may be used for fixation of osteotomies and joint arthrodeses according to the present disclosure;

FIG. 5 illustrates an exemplary embodiment of a bowed plate that may be used for fixation of osteotomies and joint arthrodeses in accordance with the present disclosure;

FIG. 6 illustrates an exemplary-use environment wherein a bowed plate is loaded into a surgical bending instrument;

FIG. 7 illustrates an exemplary-use environment wherein a bowed plate is straightened by way of a surgical bending instrument;

FIG. 8 illustrates an exemplary embodiment of a bowed plate assembly that may be used for fixation of osteotomies and joint arthrodeses according to the present disclosure;

FIG. 8A illustrates a close-up view of an exemplary embodiment of a leg comprising the bowed plate assembly of FIG. 8;

FIG. 8B illustrates a close-up view of an exemplary embodiment of a support portion comprising the bowed plate assembly of FIG. 8;

FIG. 9 illustrates an exemplary embodiment of a bowed plate assembly that includes two legs that may be used for fixation of osteotomies and joint arthrodeses;

FIG. 10 illustrates an exemplary embodiment of a bowed plate assembly that includes two legs and three additional apertures that may be used for fixation of osteotomies; and

FIG. 11 illustrates an exemplary embodiment of a bowed plate assembly that includes three legs and two additional apertures that may be used for fixation of osteotomies and joint arthrodeses according to the present disclosure.

While the present disclosure is subject to various modifications and alternative forms, specific embodiments thereof have been shown by way of example in the drawings and will herein be described in detail. The invention should be understood to not be limited to the particular forms disclosed, but on the contrary, the intention is to cover all modifications, equivalents, and alternatives falling within the spirit and scope of the present disclosure.

### DETAILED DESCRIPTION

In the following description, numerous specific details are set forth in order to provide a thorough understanding of the present disclosure. It will be apparent, however, to one of ordinary skill in the art that the invention disclosed herein may be practiced without these specific details. In other instances, specific numeric references such as “first aperture,” may be made. However, the specific numeric reference should not be interpreted as a literal sequential order but rather interpreted that the “first aperture” is different than a “second aperture.” Thus, the specific details set forth are merely exemplary. The specific details may be varied from and still be contemplated to be within the spirit and scope of the present disclosure. The term “coupled” is defined as meaning connected either directly to the component or indirectly to the component through another component. Further, as used herein, the terms “about,” “approximately,” or “substantially” for any numerical values or ranges indicate a suitable dimensional tolerance that allows the part or collection of components to function for its intended purpose as described herein.

In general, the present disclosure describes an apparatus and a method for a bowed plate for fixating osteotomies and joint arthrodeses. The bowed plate includes a backplate comprising a support portion and an aperture portion that share an intervening central portion. The central portion includes a curvature that angles the support portion and the aperture portion toward a midline of the bowed plate. A leg extends from the support portion. The leg could include one or more teeth, circumferential grooves, or threads as fixation features. These fixation features enable the leg to grip an inner wall of a hole drilled in bone. A tapered portion joins the leg to the bowed plate and provides increased strength and compression. The bowed plate is configured to be straightened by way of a surgical bending instrument for placement at a bone fixation or fusion site of a patient. Upon relaxing a degree of force applied to the bowed plate by the surgical bending instrument, the leg and the bone screw compress the fusion site.

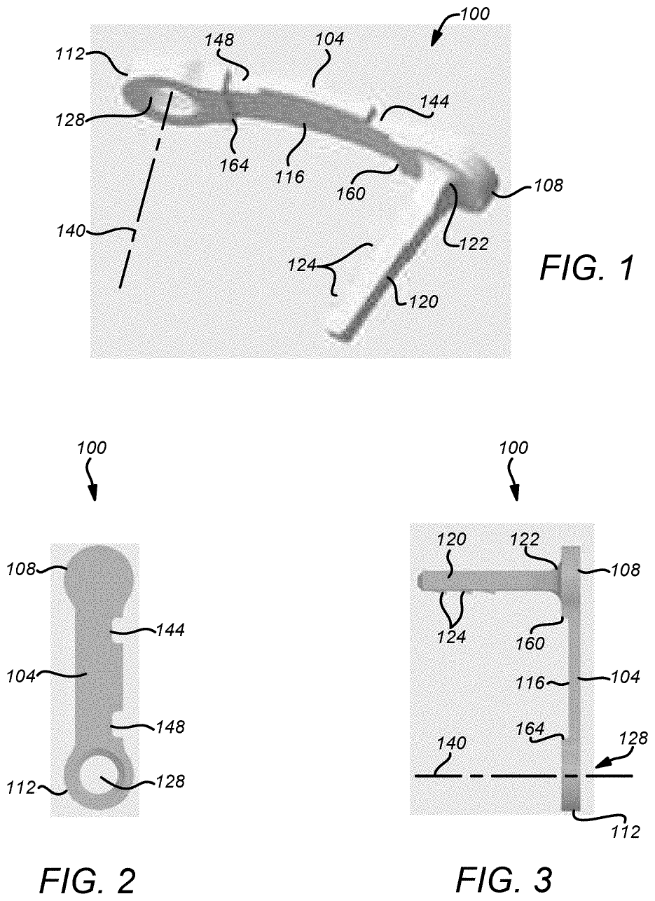

FIG. 1 illustrates an isometric view of an exemplary embodiment of a bowed plate 100 in accordance with the present disclosure. The bowed plate 100 generally is of a variety indicated for fixation of osteotomies and joint arthrodeses of the hands and feet. Upon being placed into a straightened configuration, the bowed plate 100 may be implanted at a bone fixation or fusion site of a patient, as described herein. As such, the bowed plate 100 preferably is comprised of a material that exhibits elastic properties, such as Nitinol or other similar material. The elastic properties of the material comprising the bowed plate 100 ensure that the bowed plate advantageously compresses the bones to be fused when the bowed plate 100 is allowed to return to an initial, bowed configuration illustrated in FIG. 1.

The bowed plate 100 generally comprises an elongate backplate 104 that includes a support portion 108 and an aperture portion 112 that share an intervening central portion 116. A leg 120 extends from the support portion 108. The leg 120 is similar to a leg generally comprising a surgical staple, and as such the leg 120 is adapted to be implanted into a hole drilled in a bone. In the illustrated embodiment, the leg 120 includes one or more teeth 124 that are biased toward the backplate 104 and configured to enable the leg 120 to grip an inner wall of the bone hole. In some embodiments, however, the leg 120 may include fixation features that include one or more teeth, similar to the teeth 124, circumferential grooves, threads, or any combination thereof, without limitation. Further, the leg 120 may be joined to the support portion 108 by way of a tapered portion 122, as shown in FIG. 1. It is contemplated that the tapered portion 122 generally strengthens the leg 120 with respect to the support portion 108.

As best shown in FIG. 2, the aperture portion 112 has a shape and a size that are substantially similar to the shape and size of the support portion 108. The aperture portion 112 includes a slanted aperture 128 configured to receive a bone screw 132, as shown in FIG. 7. Once the bone screw 132 is tightened into a bone hole, the slanted aperture 128 is configured to countersink a head portion (not shown) of the bone screw 132 and press the aperture portion 112 against the bone to be treated. As will be appreciated, limiting protrusion of the head of the bone screw 132 above the backplate 104 advantageously minimizes irritation to nearby soft tissue that may otherwise occur due to a relatively greater presence of the head portion.

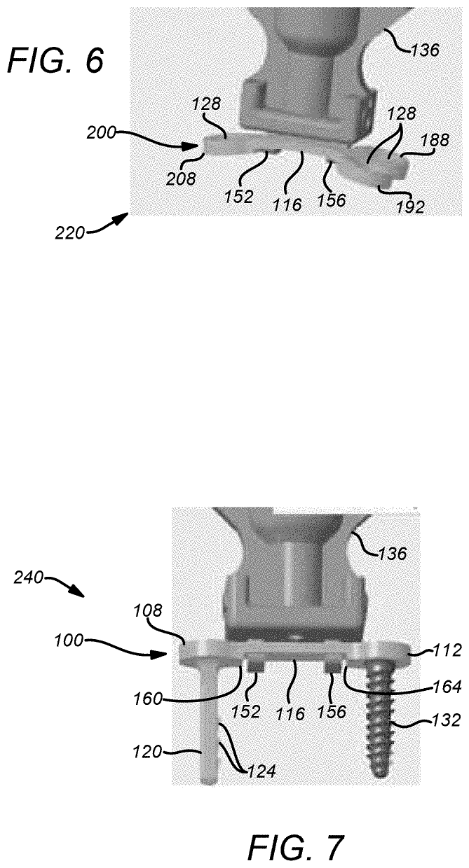

Turning again to FIG. 1, the central portion 116 is configured to be clasped by a surgical bending instrument 136 (see FIGS. 6-7) and straightened for placement at a bone fixation or fusion site of a patient, as described herein. While in the initial, bowed configuration shown in FIG. 1, the central portion 116 has a curvature that angles the leg 120 and a centerline 140 of the slanted aperture 128 toward a midline of the bowed plate 100. Upon being placed into a straightened configuration, as shown in FIG. 3, the curvature of the central portion 116 may be straightened such that the leg 120 and the centerline 140 of the slanted aperture 128 become parallel to one another and thus are suitable for implantation into the patient. It is contemplated that the leg 120 may be inserted into a first hole drilled at a fusion site of the patient, and a bone screw, such as the bone screw 132 of FIG. 7, may be inserted through the slanted aperture 128 and driven into a second hole drilled across the fusion site. The bone screw 132 may be tightened to cause the support portion 108 and the aperture portion 112 to press against the bone. When the central portion 116 is allowed to relax to the bowed configuration of FIG. 1, the leg 120 and the bone screw 132 compress the bone between the first and second drilled holes, thereby closing the fusion site. Further details pertaining to the surgical bending instrument 136, and methods for distracting implants, are disclosed in U.S. Pat. No. 10,010,320, entitled “Surgical Bending Instrument,” the entirety of which is incorporated herein by reference.

As shown in FIGS. 1-2, the central portion 116 includes a first notch 144 and a second notch 148 that are adapted to respectively accommodate a first grip 152 and a second grip 156 comprising the surgical bending instrument 136 shown in FIGS. 6-7. The notches 144, 148 serve to center the bowed plate 100 within the surgical bending instrument 136 as well as enable the grips 152, 156 to extend across a majority of the width of the backplate 104. Further, as shown in FIG. 3, the central portion 116 is bound by a first guide edge 160 and a second guide edge 164 that cooperate to center the bowed plate 100 between the grips 152, 156 of the surgical bending instrument 136. Furthermore, it is contemplated that the central portion 116 facilitates removing the grips 152, 156 from between the patient's bone and the backplate 104 after the bowed plate 100 has been tightened into the bone, as described herein.

In some embodiments, the notches 144, 148 and the guide edges 160, 164 are configured to advantageously position a driver (not shown) comprising the surgical bending instrument 136 at a location of the backplate 104 that is optimal for straightening the bowed plate 100, as shown in FIG. 7. During operation of the surgical bending instrument 136, the driver may be advanced distally toward the backplate 104 of the bowed plate 100. Once the driver contacts the optimal location of the backplate 104, the bowed plate 100 becomes clasped between the grips 152. 156 and the driver, as shown in FIG. 6. Further advancing the driver exerts an increasing degree of force onto the backplate 104 by the grips 152, 156 and the driver. As such, the distally directed force due to the driver and the proximally directed forces due to the grips 152, 156 cause a corresponding straightening of the central portion 116. Thus, operating the surgical bending instrument 136 enables a surgeon to change the bowed plate 100 from the initial configuration, shown in FIG. 1, to the straightened configuration wherein the leg 120 and the bone screw 132 are forced into a parallel relationship, as illustrated in FIG. 7.

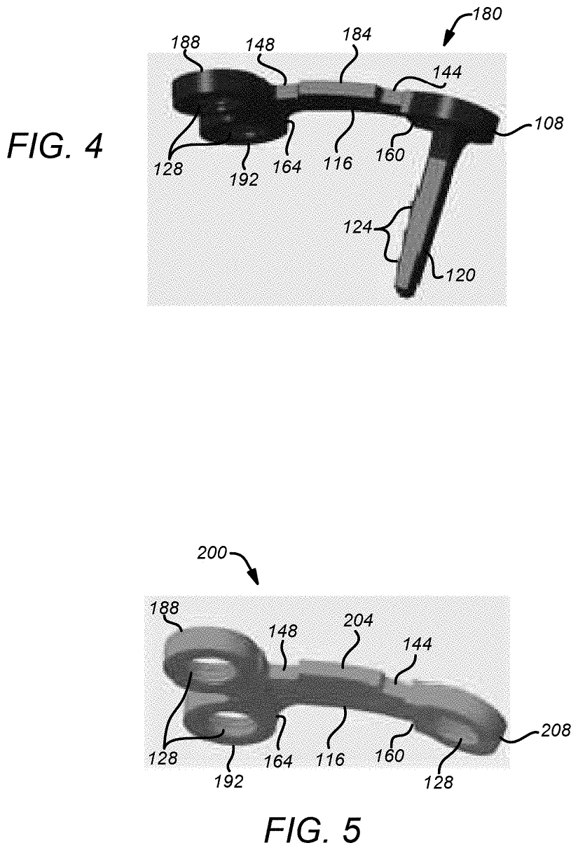

It should be borne in mind that the bowed plate 100 is not to be limited to the specific configuration shown in FIGS. 1-3 and described herein, but rather the bowed plate 100 may be adapted to accommodate various bone fixation or fusion treatments as well as different bones that may be advantageously treated by way of osteometry. For example, FIG. 4 illustrates an exemplary embodiment of a bowed plate 180 that may be used for fixation of osteotomies and joint arthrodeses. The bowed plate 180 is substantially similar to the bowed plate 100, shown in FIG. 1, with the exception that the bowed plate 180 includes a backplate 184 having a first aperture portion 188 and a second aperture portion 192. A slanted aperture 128 is disposed in each of the first and second aperture portions 188, 192. As such, the bowed plate 180 is suitable for treatments wherein two bone screws, such as the bone screw 132, are desirable in lieu of using only one bone screw.

Moreover, FIG. 5 illustrates an exemplary embodiment of a bowed plate 200 that may be used for fixation of osteotomies and joint arthrodeses. The bowed plate 200 is substantially similar to the bowed plate 180, shown in FIG. 4, with the exception that the bowed plate 200 includes a backplate 204 having a third aperture portion 208, in lieu of the support portion 108. Like the first and second aperture portions 188, 192, the third aperture portion 208 includes a slanted aperture 128, as discussed in connection with FIGS. 1-3. Thus, the bowed plate 200 may be used for treatments wherein a bone screw, such as the bone screw 132, is indicated instead of the leg 120 shown in FIG. 4.

It should be understood, however, that the slanted apertures 128 disposed in the first, second and third aperture portions 188, 192, 208 need not be the same, but rather any one or more of the aperture portions 188, 192, 208 may include differently configured slanted apertures 128, without limitation. For example, in some embodiments, one or more of the aperture portions 188, 192, 208 may be configured to receive different sizes of the bone screw 132. Further, in some embodiments, one or more of the aperture portions 188, 192, 208 may include a counterbored aperture, in lieu of the slanted aperture 128.

Moreover, in some embodiments, any one or more of the aperture portions 188, 192, 208 may be configured to include a leg, similar to the leg 120 of FIG. 1, in lieu of a slanted aperture 128. Thus, in one exemplary embodiment, the first and second aperture portions 188, 192 may be each adapted to support a leg 120 while the third aperture portion 208 is configured to receive the bone screw 132, as described herein. These and other variations of the bowed plates 100, 180, and 200, disclosed herein should be understood to be within the spirit and scope of the present disclosure, without limitation.

Turning, now, to FIG. 6, an exemplary-use environment 220 is illustrated wherein a bowed plate 200, as shown in FIG. 5, is loaded into a surgical bending instrument 136. As shown in FIG. 6, a central portion 116 comprising the bowed plate 200 is clasped between a first grip 152 and a second grip 156, and a driver (not shown) comprising the surgical bending instrument 136. Although the driver and the grips 152, 156 are in contact with the central portion 116, the force applied by the driver is sufficient to retain the central portion 116 but is not enough to straighten the bowed plate 200. As such, the bowed plate 200 is retained in the surgical bending instrument 136 in the initial, bowed configuration. However, upon advancing the driver with an increasing degree of force onto the central portion 116, the distally directed force due to the driver and proximally directed forces due to the grips 152, 156 cause a corresponding straightening of the central portion 116. Thus, operating the surgical bending instrument 136 enables a surgeon to change the bowed plate 200 from the bowed configuration, shown in FIG. 6, to a straightened configuration, as described herein.

FIG. 7 illustrates an exemplary-use environment 240 wherein a bowed plate 100 (see FIG. 1) is loaded into a surgical bending instrument 136. As shown in FIG. 7, a central portion 116 comprising the bowed plate 100 is forcibly clasped between a first grip 152 and a second grip 156, and a driver (not shown) comprising the surgical bending instrument 136. In the environment 240 of FIG. 7, the distally directed force due to the driver and the proximally directed forces due to the grips 152, 156 cause a corresponding straightening of the central portion 116. As such, a bone screw 132 that is inserted into an aperture portion 112 of the bowed plate 100 is parallel to a leg 120 protruding from a support portion 108 of the bowed plate 100. It is contemplated that the leg 120 may be inserted into a first hole drilled at a fusion site of the patient, and the bone screw 132 may be driven into a second hole drilled across the fusion site. The bone screw 132 may be tightened to press the support portion 108 and the aperture portion 112 against the bone surface. When the central portion 116 is allowed to relax to the initial, bowed configuration of FIG. 1, the leg 120 and the bone screw 132 compress the bone between the first and second drilled holes, thereby closing the fusion site. The surgical bending instrument 136 may be removed from the fusion site by removing the grips 152, 156 from between the bone surface and the central portion 116, as described herein.

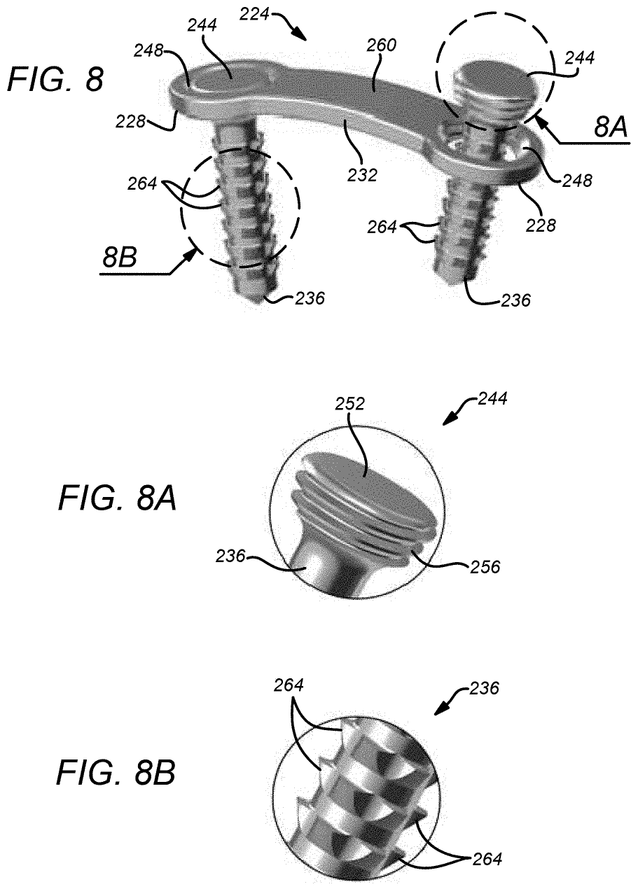

FIG. 8 illustrates an exemplary embodiment of a bowed plate assembly 224 that may be used for fixation of osteotomies and joint arthrodeses according to the present disclosure. The bowed plate assembly 224 generally comprises a pair of aperture portions 228 disposed at opposite ends of an intervening central portion 232. A leg 236 is configured to be fastened into each of the aperture portions 228, such that two legs 236 extend from the central portion 232 similar to legs comprising a surgical staple. Although the illustrated embodiment of the bowed plate assembly 224 includes two legs 236, it should be borne in mind that any suitable number of legs may be incorporated into the bowed plate assembly 224, without limitation.

As shown in FIG. 8B, each leg 236 includes a support portion 244 that is adapted to be threaded into a threaded aperture 248 disposed within each aperture portion 228. The support portion 244 includes a flat top surface 252 surrounded by tapered peripheral threads 256 that are configured to engage with threads comprising the threaded aperture 248. The threaded aperture 248 includes a taper adapted to countersink the tapered peripheral threads 256 once the flat top surface 252 is substantially flush with the top surface 260 of the central portion. In some embodiments, the support portion 244 may be welded or otherwise adhered to the threaded aperture 248 so as to fixate the support portion 244 within the aperture portion 228.

As mentioned above, the legs 236 are similar to legs comprising a surgical staple. As such the legs 236 are adapted to be implanted into a hole drilled in a bone. In the illustrated embodiment of FIG. 8A, the leg 236 includes one or more circumferential teeth 264 that are biased toward the central portion 232. The circumferential teeth 264 are configured to enable the leg 236 to grip an inner wall of the bone hole regardless of the angular disposition of the support portion 244 within the threaded aperture 248. It should be borne in mind, however, that the legs 236 illustrated and discussed herein are not limited to including solely circumferential teeth 264. In some embodiments, for example, each leg 236 may include any of various fixation features that may include one or more teeth, similar to the circumferential teeth 264, circumferential grooves, notches, wedges, threads, or any combination thereof, without limitation. It is contemplated that the various fixation features may be included in addition to or in lieu of the circumferential teeth 264, as desired.

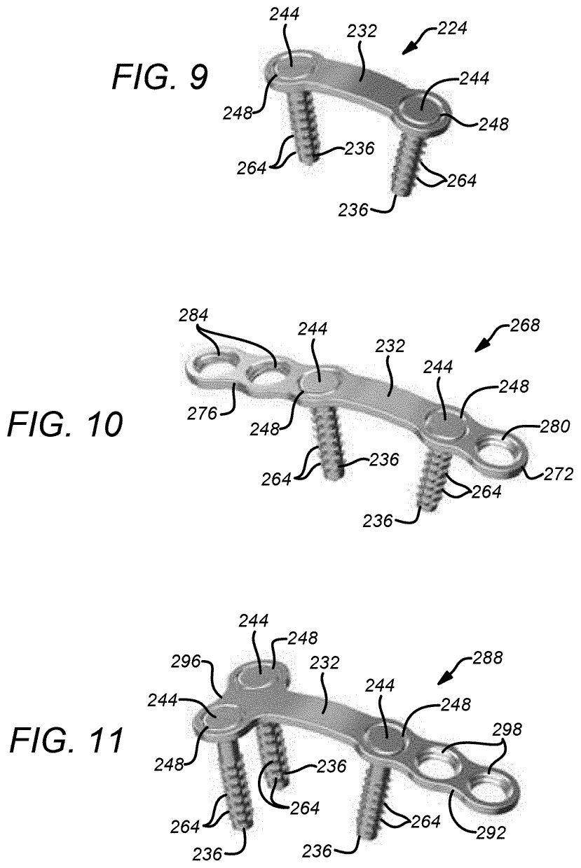

Practicing the bowed plate assembly 224 is substantially similar to practicing the bowed plate 100, shown in FIG. 1, with the exception that the bowed plate assembly 224 may be assembled before implantation into a bone fusion site. Methods for assembling the bowed plate assembly 224 include threading the support portions 244 into the threaded apertures 248, such that the legs 236 extend away from the central portion 232 as shown in FIG. 9. The legs 236 may be fixated to the central portion 232 by welding or adhering the support portions 244 within the threaded apertures 248.

Once assembled, the bowed plate assembly 224 may be loaded into a suitable surgical bending instrument, such as the surgical bending instrument 136 shown in FIG. 6. The central portion 232 of the bowed plate assembly 224 may be forcibly clasped between the first grip 152 and the second grip 156, and a driver (not shown) comprising the surgical bending instrument 136. The driver may be advanced distally to straighten the central portion 232 until the legs 236 are parallel to one another. One leg 236 may then be inserted into a first hole drilled at a fusion site of the patient, and the other leg 236 may be inserted into a second hole drilled across the fusion site. When the central portion 232 is allowed to relax to the initial, bowed configuration of FIG. 9, the legs 236 compress the bone between the first and second drilled holes, thereby closing the fusion site. The circumferential teeth 264 engage with the inner walls of the first and second drilled holes and ensure that the legs 236 remain disposed within the first and second drilled holes. The surgical bending instrument 136 may be removed from the fusion site by removing the grips 152, 156 from between the bone surface and the central portion 232.

It should be borne in mind that any suitable number of legs and fixation apertures may be incorporated into the bowed plate assembly, without limitation. For example, FIG. 10 illustrates an exemplary embodiment of a bowed plate assembly 268 that includes two legs 236 and three additional apertures that may be used for fixation of osteotomies, as described herein. The bowed plate assembly 268 includes a pair of threaded apertures 248 at opposite ends of an intervening central portion 232. A leg 236 is threadably engaged with each of the threaded apertures 248 and extending away from the central portion 232. Each of the legs 236 includes one or more circumferential teeth 264 that are biased toward the central portion 232. Further, an additional aperture portion 272 and an elongate aperture portion 276 are disposed at opposite ends of the bowed plate assembly 268. The aperture portion 272 includes a threaded aperture 280 that is configured to receive a suitable bone screw 132 (see FIG. 7) or an additional leg 236, as desired. The elongate aperture portion 276 includes two in-line threaded apertures 284 that are each configured to receive a bone screw, such as the bone screw 132, or an additional leg 236.

FIG. 11 illustrates an exemplary embodiment of a bowed plate assembly 288 that includes three legs 236 and two additional apertures that may be used for fixation of osteotomies and joint arthrodeses. The bowed plate assembly 288 includes a central portion 232 disposed between an elongate aperture portion 292 and a side-by-side aperture portion 296. The elongate aperture portion 292 includes a threaded aperture 248, and the side-by-side aperture portion 296 includes two adjacent threaded apertures 248. A leg 236 is engaged with each of the threaded apertures 248, as described hereinabove. Each of the legs 236 includes one or more circumferential teeth 264 that are biased toward the central portion 232. The elongate aperture portion 292 further includes two threaded apertures 298 that are disposed in-line with the threaded aperture 248 and the central portion 232. Either one or both of the threaded apertures 298 may be configured to receive a suitable bone screw 132 (see FIG. 7) or an additional leg 236, as desired.

While the invention has been described in terms of particular variations and illustrative figures, those of ordinary skill in the art will recognize that the invention is not limited to the variations or figures described. In addition, where methods and steps described above indicate certain events occurring in certain order, those of ordinary skill in the art will recognize that the ordering of certain steps may be modified and that such modifications are in accordance with the variations of the invention. Additionally, certain of the steps may be performed concurrently in a parallel process when possible, as well as performed sequentially as described above. To the extent there are variations of the invention, which are within the spirit of the disclosure or equivalent to the inventions found in the claims, it is the intent that this patent will cover those variations as well. Therefore, the present disclosure is to be understood as not limited by the specific embodiments described herein, but only by scope of the appended claims.

What is claimed is:

1. A bowed plate for fixating osteotomies and joint arthrodeses, the bowed plate comprising:
a backplate comprising a support portion and an aperture portion that share an intervening central portion;
a leg extending from the support portion; and
one or more fixation features disposed on the leg.

2. The bowed plate of claim 1, wherein the one or more fixation features include one or more teeth, circumferential grooves, threads, or any combination thereof.

3. The bowed plate of claim 1, wherein the bowed plate is comprised of a material that exhibits elastic properties, such as Nitinol or other metallic or polymer materials.

4. The bowed plate of claim 1, wherein the leg is adapted to be implanted into a hole drilled in a bone.

5. The bowed plate of claim 1, wherein the one or more fixation features are configured to enable the leg to grip an inner wall of a bone hole.

6. The bowed plate of claim 1, wherein the central portion includes a curvature that angles the leg and a centerline of the slanted aperture toward a midline of the bowed plate.

7. The bowed plate of claim 1, wherein the central portion is configured to be straightened by way of a surgical bending instrument for placement at a bone fixation or fusion site of a patient.

8. The bowed plate of claim 1, wherein the central portion includes a first notch and a second notch that are adapted to respectively accommodate a first grip and a second grip comprising a surgical bending instrument for straightening the bowed plate.

9. The bowed plate of claim 8, wherein the first notch and the second notch serve to center the bowed plate within the surgical bending instrument and enable the first grip and the second grip to extend across a majority of the width of the backplate.

10. The bowed plate of claim 8, wherein the central portion is configured to facilitate removing the first grip and the second grip from between a bone surface and the backplate after implantation of the bowed plate into the bone.

11. The bowed plate of claim 8, wherein the central portion is bound by a first guide edge and a second guide edge that cooperate to center the bowed plate between the first grip and the second grip of the surgical bending instrument.

12. The bowed plate of claim 11, wherein the first notch and the second notch and the first guide edge and the second guide edge are configured to position a driver of the surgical bending instrument at a location of the central portion that is optimal for straightening the bowed plate.

13. A bowed plate for fixating osteotomies and joint arthrodeses, the bowed plate comprising:
a backplate comprising a central portion having a first end and a second end;
a support portion joined with the first end;
a first aperture portion joined with the second end; and
a second aperture portion joined with the second end.

14. The bowed plate of claim 13, wherein a leg extends from the support portion and includes one or more teeth, circumferential grooves, threads, or any combination thereof.

15. The bowed plate of claim 13, wherein each of the first aperture portion and the second aperture portion includes a slanted aperture for receiving a bone screw.

16. The bowed plate of claim 13, wherein the support portion comprises a third aperture portion that includes a slanted aperture for receiving a bone screw.

17. A bowed plate assembly for fixating osteotomies and joint arthrodeses, the assembly comprising:
a central portion disposed between a first aperture portion and a second aperture portion;
one or more legs coupled with the first aperture portion; and
one or more legs coupled with the second aperture portion.

18. The assembly of claim 17, wherein any one of the one or more legs includes one or more circumferential teeth that are biased toward the central portion.

19. The assembly of claim 17, wherein any one of the one or more legs comprises a support portion that is adapted to be threaded into a threaded aperture disposed within the first aperture portion or the second aperture portion.

20. The assembly of claim 19, wherein the support portion includes a flat top surface surrounded by tapered peripheral threads that are configured to engage with threads comprising the threaded aperture.

21. The assembly of claim 17, wherein the first aperture portion includes side-by-side aperture portions.

22. The assembly of claim 17, wherein the second aperture portion includes one or more in-line aperture portions.

23. A method for fixating a bone fusion site of a patient, comprising:
drilling a first bone hole at the bone fusion site;
drilling a second bone hole across the bone fusion site;
straightening a bowed plate by way of a surgical bending instrument;
inserting a leg comprising the bowed plate into the first bone hole;
extending a bone screw through an aperture portion of the bowed plate;
tightening the bone screw into the second bone hole;
relaxing the bowed plate such that the leg and the bone screw compress the bone fusion site; and
removing the surgical bending instrument from between the bone fusion site and the bowed plate.

24. The method of claim 23, wherein inserting the leg includes aligning a slanted aperture with the second bone hole.

25. The method of claim 23, wherein tightening includes causing the aperture portion and a support portion to contact the bone fusion site.

26. The method of claim 23, wherein relaxing includes reducing a degree of force applied to the bowed plate by the surgical bending instrument.

27. The method of claim 23, wherein straightening includes:
loading the bowed plate into the surgical bending instrument;
activating the surgical bending instrument to forcibly clasp the bowed plate; and
operating the surgical bending instrument to manipulate the bowed plate from a bowed configuration to a straightened configuration.
